(19) United States
(12) Patent Application Publication
(10) Pub. No.: US 2020/0246485 A1
(43) Pub. Date: Aug. 6, 2020

(54) METHOD FOR GENERATING A DIHYDROMYRICETIN (DHM) FORMULATION

(71) Applicant: 82 LABS INC, Los Angeles, CA (US)

(72) Inventor: Fu Chen, Hacienda Heights, CA (US)

(21) Appl. No.: 15/929,100

(22) Filed: Jan. 31, 2019

(51) Int. Cl. A61K 47/69; A61K 47/12; A61K 47/22; A61K 47/26; A61K 31/352; A61K 9/08

(52) U.S. Cl. CPC A61K 47/6951; A61K 47/12; A61K 47/22; A61P 25/32; A61K 31/352; A61K 9/08; A61K 47/26

(57) ABSTRACT

A method of generating a soluble dihydromyricetin formulation may involve generating an acidic solution with a pH ranging between 2-7 pH in a reaction vessel by combining an organic acid in an aqueous solution at a first temperature range. The method may then generate a solvent mixture by combining the acidic solution with a sugar. The method may then heat the solvent mixture to a boil and then adjust the heat to the first temperature range generating a heated solvent mixture. The method may then combine cyclodextrin and dihydromyricetin (DHM) with the heated solvent mixture forming a DHM solution. The method may then mix the DHM solution at a first speed for a first time interval in a mixer. The method may then heat a mixed DHM solution at a second temperature range for a second time interval.

### BACKGROUND

Dihydromyricetin (DHM) is a flavanonol with antioxidant and anti-cancer activity, found to have anti-alcohol intoxication effects. Its anti-alcohol effects appear to be by its actions as a positive modulator of GABA-A receptors at the benzodiazepine site. In some studies, administration of DHM counteracted acute alcohol intoxication, and also withdrawal symptoms including tolerance, increased anxiety and seizure susceptibility. Despite its therapeutic promise, DHM is faced with the problem of low oral bioavailability. The low bioavailability is presumably caused by the combined effects of its low solubility (0.2 mg/mL at 25° C.) and poor permeability (P_{eff}=(1.84±0.37)×10₋₆ cm/s). Therefore, a need exists to improve the bioavailability of the DHM.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a chemical structure of an organic molecule 100 corresponding to dihydromyricetin in accordance with one embodiment.

FIG. 2 illustrates a chemical structure of organic molecule 200 corresponding to sucralose in accordance with one embodiment.

FIG. 3 illustrates chemical structure of organic molecules 300 corresponding to cyclodextrins in accordance with one embodiment.

FIG. 4 illustrates a system 400 in accordance with one embodiment.

FIG. 5 illustrates a reaction 500 in accordance with one embodiment.

FIG. 6 illustrates a method 600 in accordance with one embodiment.

### DETAILED DESCRIPTION

A method of generating a soluble dihydromyricetin formulation may involve generating an acidic solution with a pH ranging between about 2-7 pH in a reaction vessel by combining an organic acid ranging between about 0.1-5.0 w % of the formulation, in an aqueous solution ranging between about 80-99 w % of the formulation, at a first temperature range. The method may generate a solvent mixture by combining the acidic solution with a sugar ranging between about 1.0-20.0 w % of the formulation, in the reaction vessel. The method may apply heat to the solvent mixture in the reaction vessel raising it to a boil and then adjust the heat to the first temperature range generating a heated solvent mixture. The method may combine sugar ranging between about 1.0-20.0 w % of the formulation, and dihydromyricetin (DHM) ranging between about 0.1-3.0 w % of the formulation, with the heated solvent mixture in the reaction vessel forming a DHM solution. The method may mix the DHM solution at a first speed for a first time interval ranging between about 8-12 minutes in a mixer. The method may heat the mixed DHM solution at a second temperature range ranging between about 80-90° C. in the reaction vessel for a second time interval.

In some configurations, the sugar is at least one of a monosaccharide, a disaccharide, a polysaccharide, sugar substitute, sugar alcohol and combinations thereof. The sugar substitute may be steviol glycoside or sucralose. The sugar alcohol may be xylitol.

In some configurations, the organic acid may comprise citric acid ranging between about 0.1-1.0 w % and ascorbic acid ranging between about 0.5-1.5 w %.

In some configurations, the first speed ranges between about 2000-3000 rotations per minute (RPM) of the mixer.

In some configurations, the second time interval ranges between about 5-15 minutes.

In some configurations, the mixed DHM solution comprises DHM-cyclodextrin complexes.

In some configurations, the cyclodextrin may be a β-Cyclodextrin.

In some configurations, the first temperature range may be between about 85-90° C.

A system for generating a soluble dihydromyricetin formulation may include a reaction vessel, a heating element, and a mixer.

The reaction vessel may be configured to generate an acidic solution with a pH ranging between about 2-7 pH by combining an organic acid ranging between about 0.1-5.0 w % of the formulation, in an aqueous solution ranging between about 80-99 w % of the formulation, at a first temperature range. the reaction vessel may be configured to generate a solvent mixture by combining the acidic solution with a sugar ranging between about 1.0-20.0 w % of the formulation. The reaction vessel may be configured to generate a heated solvent mixture by receiving heat from the heating element raising the solvent mixture to a boil and then returning to the first temperature range. The reaction vessel may be configured to combine sugar ranging between about 1.0 -20.0 w % of the formulation, and dihydromyricetin (DHM) ranging between about 0.1-3.0 w % of the formulation, with the heated solvent mixture in the reaction vessel forming a DHM solution.

The mixer being configured to mix the DHM solution at a first speed for a first time interval ranging between about 8-12 minutes in a mixer; and

The heating element being configured to apply heat to the solvent mixture in the reaction vessel raising it to a boil and then adjust the heat to the first temperature range generating the heated solvent mixture. The heating element may be configured to heat the mixed DHM solution at a second temperature range ranging between about 80-90° C. in the reaction vessel for a second time interval.

In some configurations, the sugar is at least one of a monosaccharide, a disaccharide, a polysaccharide, sugar substitute, sugar alcohol and combinations thereof. The sugar may be sucralose. The sugar substitute may be steviol glycoside. The sugar alcohol may be xylitol.

In some configurations, the organic acid comprises citric acid ranging between about 0.1-1.0 w % and ascorbic acid ranging between about 0.5-1.5 w %.

In some configurations, the first speed ranges between about 2000-3000 rotations per minute (RPM) of the mixer.

In some configurations, the second time interval ranges between about 5-15 minutes.

In some configurations, the mixed DHM solution may comprise DHM-cyclodextrin complexes.

In some configurations, the cyclodextrin may be a β-Cyclodextrin.

In some configurations, the first temperature range may be between about 85-90° C.

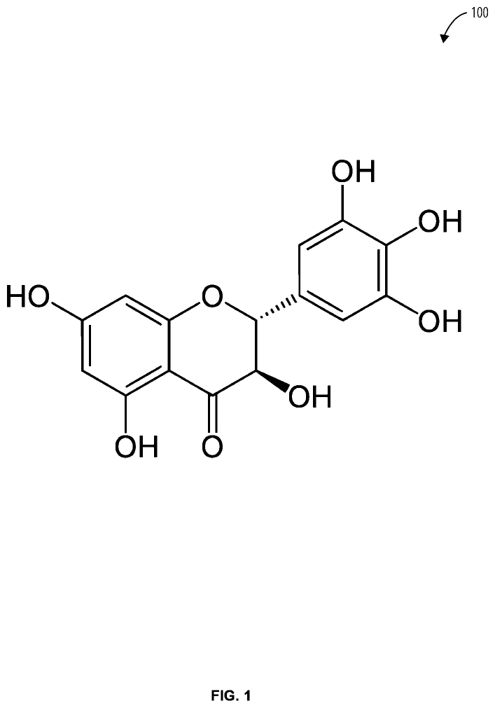

FIG. 1 illustrates a chemical structure of an organic molecule 100 corresponding to dihydromyricetin. Dihydromyricetin (DHM, (2R,3R)-3,5,7-trihydroxy-2-(3,4,5-trihydroxyphenyl)-2,3-dihydrochromen-4-one)) is a flavanonol with antioxidant and anti-cancer activity, found to have anti-alcohol intoxication effects. Its anti-alcohol effects appear to be by its actions as a positive modulator of GABA-A receptors at the benzodiazepine site.

DHM can be found in Ampelopsis Grossedentata (Snake Wine Vine, also called Vine Tea or Teng Cha in China), Japanese Raisin Tree (Hovenia Dulcis), the Himilayan Cedar Tree (Cedrus Deodara) or the African Blackwood (Erythrophleum Africanum). DHM may be extracted through an ethanol extraction.

TABLE 1
DHM Solubility in Water

| Temp (° C.) | Solubility (mg/ml) |
|---|---|
| 25° C. | 0.7 |
| 100° C. | 15.5 |

As seen in Table 1, DHM has a solubility of 0.7 mg/ml at room temperature (˜25° C.). However, the solubility of DHM improves in hot water (˜100 ° C.) to 16.0 mg/ml.

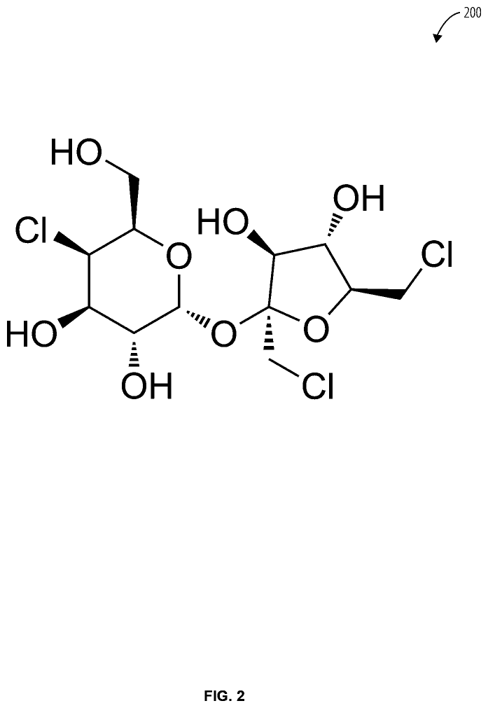

FIG. 2 illustrates a chemical structure of organic molecule 200 corresponding to sucralose (Trichlorosucrose). sucralose is a disaccharide derivative consisting of 4-chloro-4-deoxy-alpha-D-galactopyranose and 1,6-dichloro-1,6-dideoxy-beta-D-fructofuranose units linked by a glycosidic bond. It is a disaccharide derivative and an organochlorine compound and is freely soluble in water.

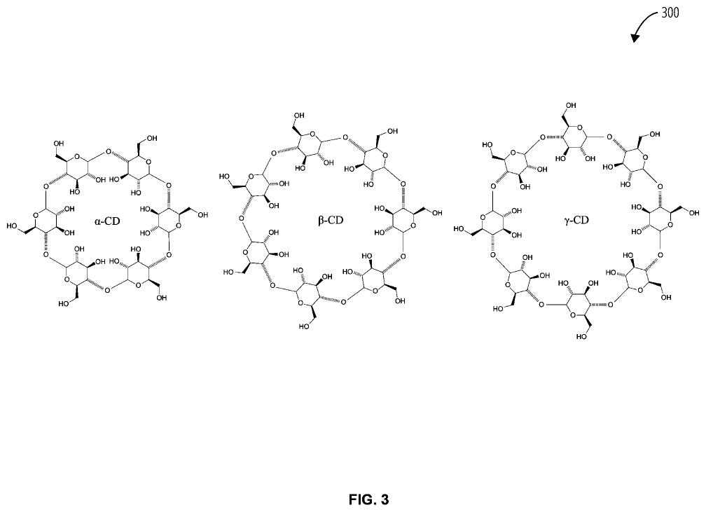

FIG. 3 illustrates chemical structure of organic molecules 300 corresponding to cyclodextrins.

Cyclodextrins are cyclic oligosaccharides with a defined number of d-glucose monomers in the ring. All glucopyranose units are linked by glycosidic α(1→4) bonds.

The three most important cyclodextrins (CDs) consist of 6, 7, or 8 glucose units and are named α-cyclodextrin, β-cyclodextrin, and γ-cyclodextrin, respectively. Higher homologues have been described but have gained no industrial significance up to now.

The three-dimensional structure of cyclodextrins resembles a truncated cone with a hydrophobic cavity. All secondary hydroxyl groups at C2 and C3 are directed towards the wider opening of the cavity, whereas the primary hydroxyl groups at C6 are located on the narrow opening. The C—H groups and a ring of glycosidic oxygen bonds are directed inside the cavity causing its hydrophobic character.

TABLE 2

| | α-Cyclodextrin | β-Cyclodextrin | γ-Cyclodextrin |
|---|---|---|---|
| Formula | C₃₆H₆₀O₃₀ | C₄₂H₇₀O₃₅ | C₄₈H₈₀O₄₀ |
| Molecular mass | 972.85 | 1135.00 | 1297.14 |
| Solubility in water | 14.5 | 1.85 | 23.2 |
| (25° C.), g/100 mL | | | |
| Crystal water, | 10.2 | 13.2-14.5 | 8.13-17.7 |
| wt % | | | |
| α_{D}²⁰, ° | +148 | +162 | +177 |
| mp, ° C. | >200° C. | >200° C. | >200° C. |
| pKₐ value (25° C.) | 12.331 | 12.202 | 12.081 |

Table 2 illustrates the physiochemical properties of cyclodextrins. Cyclodextrins are insoluble in alcohols, ketones, ethers, chlorinated hydrocarbons, and aliphatic and aromatic hydrocarbons.

Cyclodextrins are chiral, nonreducing oligosaccharides. Upon oxidation with periodate the glucose rings are cleaved; neither formic acid nor formaldehyde is produced. The only degradation product of all cyclodextrins in acidic solution is glucose. The hydrolysis rate follows the order γ>β>α. Under acidic conditions cyclodextrins are hydrolyzed more slowly than maltooligosaccharides. The glycosidic bond in cyclodextrins is hydrolyzed by α-amylase but not by β-amylase. The rate of enzymatic hydrolysis is fastest with γ-CD, followed by β-CD and α-CD. All cyclodextrins are very stable and highly soluble in alkaline solution (pH>14). Further, the solubility in water may be highly increased in basic solutions. Under nitrogen atmosphere cyclodextrins are stable up to 250° C.

Substitution of hydrogen of the primary and secondary hydroxyl groups leads to cyclodextrin derivatives. Most reactions are carried out in aqueous solutions. Other suitable solvents are dimethyl sulfoxide, dimethyl formamide, and pyridine.

Cyclodextrins are prepared by enzymatic degradation of starch with cyclodextrin glycosyltransferase (CGTase) at 30-90 ° C. For industrial production corn and potato starch are most important, but wheat and tapioca starch can also be used. Several microorganisms are able to produce CGTases, for example Bacillus macerans, Bacillus megaterium, Bacillus subtilis, Bacillus firmus, Bacillus circulans, Klebsiella pneumoniae, or Klebsiella oxytoca.

The aqueous solution of starch is converted to cyclodextrins at a temperature of 30-90° C.

Generally, CGTase enzymes produce all three major types of cyclodextrins, depending on the microbiological source of the enzyme and the conversion conditions. To improve the yield and to alter the ratio of the different cyclodextrins obtained, complexing agents (see Table 2) can be added. Most of the complexing agents form insoluble solid complexes with a certain cyclodextrin. The complex formation shifts the reaction equilibrium towards the desired cyclodextrin and the complex can be separated easily from the liquid conversion mixture. For isolation of the cyclodextrins the complex is dissociated by steam distillation or extraction of the complexing agent with an organic solvent.

The yield can be improved further by utilization of specific enzymes. For example, alpha-CGTase produced by Klebsiella oxytoca produces predominantly α-cyclodextrin in the initial phase. For large-scale production of γ-cyclodextrin a specific gamma-CGTase was developed.

The hydrophobic cavity in cyclodextrins is capable to incorporate guest molecules. With this process of molecular encapsulation, a cyclodextrin inclusion complex (adduct, clathrate) is formed. During the association no covalent or ionic bonds are formed. In solution complex formation and dissociation are in a dynamic equilibrium.

For a complex of 1:1 stoichiometry the system can be described in equation 1 as:

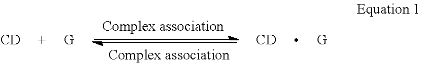

The rate constant kₐ for complex association is in the range of 10⁰-10⁸ mol/s. The complex association therefore is usually a fast process, proceeding within milliseconds. The complex formation is an exothermic reaction (ΔH <0). The complex stability decreases with increasing temperature.

An important aspect for the formation of cyclodextrin inclusion complexes is the geometry of the guest molecule. The size of a guest molecule must be compatible with the diameter of the cavity, which is dependent on the number of glucose units in the ring. With larger molecules it has to be taken into consideration that only side groups penetrate into the cyclodextrin cavity. In these cases, also complexes with higher stoichiometries towards cyclodextrins can be formed. The dependence of the ability to form inclusion complexes, as indicated by a “+”, with α-, β-, or γ-cyclodextrin on the size of the guest molecules is depicted in Table 3.

TABLE 3

| Substance | α-CD | β-CD | γ-CD |
|---|---|---|---|
| Xenon | + | — | — |
| Chlorine | + | — | — |
| Bromine | + | + | |
| Iodine | + | + | + |
| Carbon dioxide | + | — | — |
| Ethylene | + | — | — |
| Propionic acid | + | — | — |
| Butyric acid | + | + | — |
| Cyclohexane | + | + | + |
| Naphthalene | — | + | + |
| Anthracene | — | — | + |

The driving forces for complex formation may include van der Waals forces, hydrophobic interactions, change in solvation energy for both components and, to a lesser extent, hydrogen bonds. The stability of the complex therefore corresponds to the hydrophobic character of the guest molecule. Very polar or ionic molecules may form only weak complexes.

Most preparations of cyclodextrin complexes are carried out in aqueous systems. Complex formation in the absence of a solvent is too slow.

For complexation in a solution, the aqueous solutions of cyclodextrins are agitated with stoichiometric amounts of a guest substance at slightly elevated temperatures (40-60° C.) for several hours. After the equilibrium is reached water can be removed by freeze drying or spray drying. With many hydrophobic compounds and unmodified cyclodextrins insoluble complexes are obtained which can be isolated by filtration. Drying under vacuum is applied to obtain dry complexes. In some cases, the use of polar cosolvents like methanol or ethanol can be of advantage.

For complexation in a suspension, it is not necessary to work with completely dissolved cyclodextrins. Fast complexation can be achieved in stirred aqueous slurries of cyclodextrins and guest substance.

For complexation by kneading, the water content is further reduced so that the mixture of cyclodextrin, guest, and water forms a paste. To complete complexation this paste is kneaded for 30-200 min. Complexation time is usually lower than in the aforementioned methods.

For complexation with a highly soluble cyclodextrin derivatives, concentrated aqueous solutions of cyclodextrins are stirred with an excess of guest substance. In many cases the formed complexes are very soluble so that uncomplexed material can be removed by filtration. The complex solutions can be used directly or can be spray dried to obtain a water soluble powder.

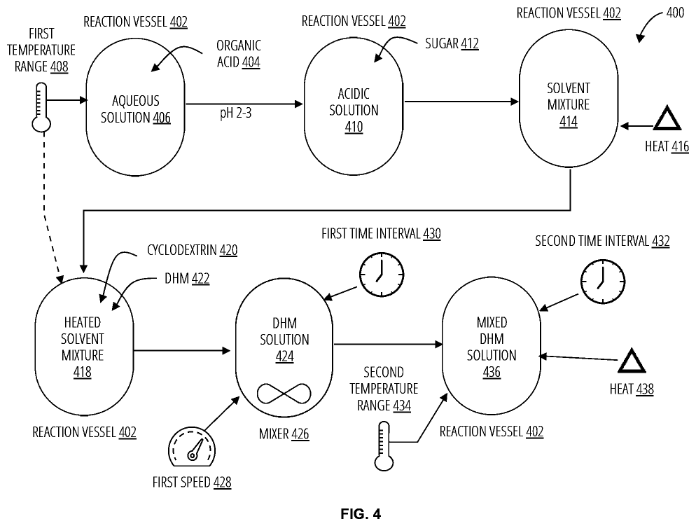

FIG. 4 illustrates a system 400 for generating a dihydromyricetin formulation. The system 400 comprises at least a reaction vessel 402, a mixer 426, and a heating element that applies heat to the reaction vessel 402. The method of generating the soluble dihydromyricetin formulation involves, combining the organic acid 404 with an aqueous solution 406 in the reaction vessel 402 at a first temperature range 408 to generate an acidic solution 410 with a pH ranging between about 2.0-7.0. A sugar 412 is then combined with the acidic solution 410 to generate a solvent mixture 414. A source of heat 416 is applied to the reaction vessel 402 with the solvent mixture 414 until the solvent mixture 414 begins to boil and then allowed to return to the first temperature range 408, thereby generating the heated solvent mixture 418. The DHM 422 and the cyclodextrin 420 are then added to the heated solvent mixture 418 forming the DHM solution 424. The DHM solution 424 is transferred to the mixer 426 where it is mixed at a first speed 428 for a first time interval 430, thereby generating the mixed DHM solution 436. The mixed DHM solution 436 is then transferred back to the reaction vessel 402 where heat 438 is applied to the reaction vessel until the mixed DHM solution 436 reaches a second temperature range 434 where it is maintained at the second temperature range 434 for a second time interval 432.

In some configurations, the heating element may apply heat directly to the contents of the reaction vessel (e.g., integrated heating elements) or indirectly heat the contents of the reaction vessel by way of heating the reaction vessel (e.g., heating plate, etc.,).

TABLE 4
Soluble Dihydromyricetin Formulation

| Compound | Range (w %) |
|---|---|
| Organic Acid | 0.1-5.0 w % |
| Aqueous Solution | 80.0-99.0 w % |
| Sugar | 1.0-20.0 w % |
| Cyclodextrin | 1.0-10.0 w % |
| Dihydromyricetin | 0.1-3.0 w % |

Table 4 illustrates one embodiment of the soluble dihydromyricetin formulation with the ranges of the ingredients as percent weight (w %) of the total formulation. The organic acid may be found ranging between about 0.1-5.0 w % of the formulation. The organic acid may be citric acid as well as other salt forms of citrate or other pH adjusting ingredients such as ascorbic acid. The aqueous solution may be found ranging between about 80.0-99.0 w % of the formulation. The sugar is present in an amount ranging between about 1.0-20.0 w % of the formulation. The sugar may be sucrose or another sugar such as glucose, fructose, etc., or sugar substitute such as xylitol, sucralose, or steviol glycoside, etc. The dihydromyricetin may be founding ranging between about 0.1-3.0 w % of the formulation. Cyclodextrin may be found ranging between about 1.0-10.0 w % of the formulation. Cyclodextrin may be β-Cyclodextrin. The formulations comprising the DHM-cyclodextrin complexes enhance the dissolution rate and in vivo bioavailability of dihydromyricetin.

In some embodiments, the soluble dihydromyricetin formulation may include additional ingredients that may supply and/or enhance therapeutic effects of the soluble dihydromyricetin formulation.

The additional ingredients may include N-Acetyle-1-Cystine, Milk Thistle Extract, Herb Mixture E, Trisodium Citric Acid, Magnesium Chloride, Calcium Lactate, Potassium Chloride, Vitamin B12 0.1% (methylcobalamin), Vitamin B1 Thiamine HCl, Vitamine B3 (Niacin), Vitamin B6 HCl, B9 (Folic Acid), Prickly Pear Concentrate, Pear Concentrate, Green Tea Concentrate, Korean Red Ginseng Extract, Hovenia Gulcis Thub Concentrate, and Natural lemon Flavor.

TABLE 5
Enhanced Formulation # 1

| Ingredients | Quantity (w %) |
|---|---|
| Purified water | 92.62310 |
| Citric Acid Anhydrous | 0.12000 |
| Vitamin C | 0.30000 |
| DHM(XS) | 0.39500 |
| Beta-Cyclodextrin | 2.00000 |
| REBATEN 97% (i.e., steviol glycosides) | 0.05500 |
| Xylitol | 2.00000 |
| Additional Ingredients | 2.50690 |
| N-Acetyle-l-Cystine | 0.14700 |
| Milk Thistle Extract | 0.07400 |
| Herb Mixture E | 0.03000 |
| Trisodium Citric Acid | 0.04000 |
| Magnesium Chloride | 0.47000 |
| Calcium Lactate | 0.78000 |
| Potassium Chloride | 0.13000 |
| Vitamin B12 0.1% (methylcobalamin) | 0.15200 |
| Vitamin B1 Thiamine HCI | 0.00850 |
| Vitamine B3 (Niacin) | 0.02850 |
| Vitamin B6 HCI | 0.00610 |
| B9 (Folic Acid) | 0.00080 |
| Prickly Pear Concentrate | 0.15000 |
| Green Tea Concentrate | 0.02500 |
| Korean Red Ginseng Extract | 0.02500 |
| Hovenia Gulcis Thub Concentrate | 0.04000 |
| Natural lemon Flavor | 0.40000 |

Table 5 illustrates one embodiment of the soluble dihydromyricetin formulation with the additional ingredients (Enhanced formulation #1) and comprising steviol glycoside and xylitol as the sugar. In this configuration, the additional ingredients total about 2.5 w % (2.50690) of the formulation.

TABLE 6
Enhanced Formulation # 2

| Ingredients | Quantity (w %) |
|---|---|
| Purified water | 84.70880 |
| Citric Acid Anhydrous | 0.15000 |
| Vitamin C | 0.30000 |
| DHM(XS) | 0.38500 |
| Beta-Cyclodextrin | 2.00000 |
| White Sugar (i.e. Sucrose) | 8.52000 |
| Additional Ingredients | 3.93620 |
| N-Acetyle-I-Cystine | 0.14300 |
| Milk Thistle Extract | 0.07200 |
| Herb Mixture E | 0.03000 |
| Trisodium Citric Acid | 0.04000 |
| Magnesium Chloride | 0.45000 |
| Calcium Lactate | 0.76000 |
| Potassium Chloride | 0.13000 |
| Vitamin B12 0.1% (methylcobalamin) | 0.15000 |
| Vitamin B1 Thiamine HCI | 0.00840 |
| Vitamine B3 (Niacin) | 0.02800 |
| Vitamin B6 HCI | 0.00600 |
| B9 (Folic Acid) | 0.00080 |
| Prickly Pear Concentrate | 0.14800 |
| Pear Concentrate | 1.48000 |
| Green Tea Concentrate | 0.02500 |
| Korean Red Ginseng Extract | 0.02500 |
| Hovenia Gulcis Thub Concentrate | 0.04000 |
| Natural lemon Flavor | 0.40000 |

Table 6 illustrates one embodiment of the soluble dihydromyricetin formulation with the additional ingredients (Enhanced formulation #2) and comprising sucrose as the sugar. In this configuration, the additional ingredients total about 4.0 w % (3.93620) of the formulation.

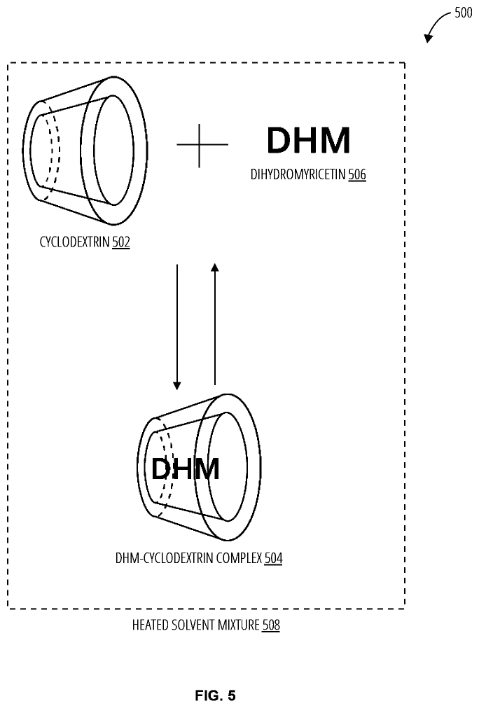

FIG. 5 shows reaction 500 illustrating the complex association between dihydromyricetin 506 and cyclodextrin 502 forming the DHM-cyclodextrin complex 504. The complex association occurs within the heated solvent mixture 508. The hydrophobic cavity in cyclodextrin 502 incorporates dihydromyricetin 506. With this process of molecular encapsulation, a cyclodextrin inclusion complex (DHM-cyclodextrin complex 504) is formed. During the association no covalent or ionic bonds are formed. In solution, complex formation and dissociation are in a dynamic equilibrium. The rate constant kₐ for complex association is in the range of 10⁰-10⁸ mol/s in water. The complex association therefore is usually a fast process, proceeding within milliseconds.

The presence of the sugar in the heated solvent mixture 508 may shift the equilibrium of the complexing towards the DHM-cyclodextrin complex 504.

TABLE 7
Process Solubility Comparison

| | DHM |
|---|---|
| | Solubility |
| Process Types | (mg/ml) |
| Unprocessed DHM in Aqueous. Solution at 25° C. | 0.70 |
| Formulation Process without β-Cyclodextrin and Heat | 15.5 |
| Soluble DHM Formulation Process | 36.1 |

Table 7 illustrates a solubility comparison of DHM having undergone the method of generating a soluble DHM formulation (Soluble DHM formulation process), a variation of the process without cyclodextrin and heat, and DHM at 25° C. At 25° C., DHM has a solubility of 0.70 mg/ml. In the formulation process that excludes the addition of β-Cyclodextrin and heat, the DHM has a solubility of 15.5 mg/ml. This improvement in solubility may be attributed to the pH of the formulation due to presence of the organic acid. However, DHM having undergone the soluble DHM formulation process, has formed DHM-cyclodextrin complexes and has a solubility of 36.1 mg/ml.

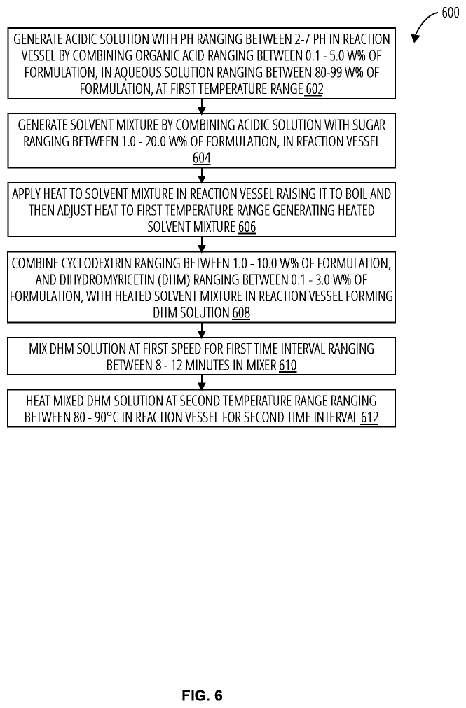

Referencing FIG. 6, a method 600 for generating a dihydromyricetin formulation involves generating an acidic solution with a pH ranging between about 2-7 pH in a reaction vessel by combining an organic acid ranging between about 0.1-5.0 w % of the formulation, in an aqueous solution ranging between about 80-99 w % of the formulation, at a first temperature range (block 602). In block 604, the method 600 generates a solvent mixture by combining the acidic solution with a sugar ranging between about 1.0-20.0 w % of the formulation, in the reaction vessel. In block 606, the method 600 applies heat to the solvent mixture in the reaction vessel raising it to a boil and then adjust the heat to the first temperature range generating a heated solvent mixture. In block 608, the method 600 combines sugar ranging between about 1.0-20.0 w % of the formulation, and dihydromyricetin (DHM) ranging between about 0.1-3.0 w % of the formulation, with the heated solvent mixture in the reaction vessel forming a DHM solution. In block 610, the method 600 mixes the DHM solution at a first speed for a first time interval ranging between about 8-12 minutes in a mixer. In block 612, the method 600 heats a mixed DHM solution at a second temperature range ranging between about 80-90° C. in the reaction vessel for a second time interval.

The methods and systems in this disclosure are described in the preceding on the basis of several preferred embodiments. Different aspects of different variants are considered to be described in combination with each other such that all combinations that upon reading by a skilled person in the field on the basis of this document may be regarded as being read within the concept of the invention. The preferred embodiments do not limit the extent of protection of this document.

Having thus described embodiments of the present invention of the present application in detail and by reference to illustrative embodiments thereof, it will be apparent that modifications and variations are possible without departing from the scope of the present invention.

1. A method of generating a soluble dihydromyricetin formulation, the method comprising:
generating an acidic solution with a pH ranging between 2-7 pH in a reaction vessel by combining an organic acid ranging between about 0.1-5.0 w % of the formulation, in an aqueous solution ranging between about 80-99 w % of the formulation, at a first temperature range;
generating a solvent mixture by combining the acidic solution with a sugar ranging between about 1.0-20.0 w % of the formulation, in the reaction vessel;
applying heat to the solvent mixture in the reaction vessel raising it to a boil and then adjust the heat to the first temperature range generating a heated solvent mixture;
combining cyclodextrin ranging between about 1.0-10.0 w % of the formulation, and dihydromyricetin (DHM) ranging between about 0.1-3.0 w % of the formulation, with the heated solvent mixture in the reaction vessel forming a DHM solution;
mixing the DHM solution at a first speed for a first time interval ranging between about 8-12 minutes in a mixer; and
heating a mixed DHM solution at a second temperature range ranging between about 80-90° C. in the reaction vessel for a second time interval.

2. The method of claim 1, wherein the sugar is at least one of a monosaccharide, a disaccharide, a polysaccharide, sugar substitute, sugar alcohol and combinations thereof.

3. The method of claim 2, wherein the sugar substitute is sucralose.

4. The method of claim 2, wherein the sugar substitute is a steviol glycoside.

5. The method of claim 2, wherein the sugar alcohol is xylitol.

6. The method of claim 1, wherein the organic acid comprises citric acid ranging between about 0.1-1.0 w % and ascorbic acid ranging between about 0.5-1.5 w %.

7. The method of claim 1, wherein the second time interval ranges between about 5-15 minutes.

8. The method of claim 1, wherein the mixed DHM solution comprises DHM-cyclodextrin complexes.

9. The method of claim 1, wherein the cyclodextrin is a β-Cyclodextrin.

10. The method of claim 1, wherein the first temperature range is between about 85-90° C.

11. A system for generating a soluble dihydromyricetin formulation comprising:
a reaction vessel;
a heating element;
a mixer;
the reaction vessel being configured to:
generate an acidic solution with a pH ranging between about 2-7 pH by combining an organic acid ranging between about 1.0-5.0 w % of the formulation, in an aqueous solution ranging between about 80-99 w % of the formulation, at a first temperature range; and
generate a solvent mixture by combining the acidic solution with a sugar ranging between about 1.0-20.0 w % of the formulation;
generate a heated solvent mixture by receiving heat from the heating element raising the solvent mixture to a boil and then returning to the first temperature range;
combine cyclodextrin ranging between about 1.0-10.0 w % of the formulation, and dihydromyricetin (DHM) ranging between about 0.1-3.0 w % of the formulation, with the heated solvent mixture in the reaction vessel forming a DHM solution; and
the mixer being configured to mix the DHM solution at a first speed for a first time interval ranging between about 8-12 minutes in a mixer; and
the heating element being configured to:
apply heat to the solvent mixture in the reaction vessel raising it to a boil and then adjust the heat to the first temperature range generating the heated solvent mixture; and
heat the mixed DHM solution at a second temperature range ranging between about 80-90° C. in the reaction vessel for a second time interval.

12. The system of claim 11, wherein the sugar is at least one of a monosaccharide, a disaccharide, a polysaccharide, sugar substitute, sugar alcohol, and combinations thereof.

13. The system of claim 12, wherein the sugar substitute is sucralose.

14. The system of claim 12, wherein the sugar substitute is a steviol glycoside.

15. The system of claim 12, wherein the sugar alcohol is xylitol.

16. The system of claim 11, wherein the organic acid comprises citric acid ranging between about 0.1-1.0 w % and ascorbic acid ranging between about 0.5-1.5 w %.

17. The system of claim 11, wherein the second time interval ranges between 5-15 minutes.

18. The system of claim 11, wherein the mixed DHM solution comprises DHM-cyclodextrin complexes.

19. The system of claim 11, wherein the cyclodextrin is a β-Cyclodextrin.

20. The system of claim 11, wherein the first temperature range is between about 85-90° C.
